(19) United States
(12) Patent Application Publication
(10) Pub. No.: US 2025/0127418 A1
(43) Pub. Date: Apr. 24, 2025

(54) DEVICE FOR ENDOSCOPIC IMAGING FOR ENDOSCOPIC PROCEDURES

(71) Applicant: ColoWrap, LLC., Durham, NC (US)

(72) Inventor: James HATHORN, Carrboro, NC (US)

(21) Appl. No.: 18/924,923

(22) Filed: Oct. 23, 2024

Related U.S. Application Data
(60) Provisional application No. 63/592,452, filed on Oct. 23, 2023.

(51) Int. Cl. A61B 5/06; A61B 1/06; A61B 1/31; A61B 17/02; A61B 34/20

(52) U.S. Cl. CPC A61B 5/066; A61B 1/0684; A61B 1/31; A61B 5/062; A61B 17/0281; A61B 34/20; A61B 2034/2055

(57) ABSTRACT

A method and apparatus for imaging of a scope during an endoscopy procedure. The apparatus comprises an endoscopy device configured for placement at or around an abdomen of a patient during the endoscopic procedure; one or more sensors comprised in the endoscopy device and configured to obtain or provide information related to positioning of an endoscope in relation with the front portion of the endoscopy device or with a set of anatomical landmarks of the patient; and a visual indication component that changes visual output relative to a position of an endoscope.

### CROSS REFERENCE TO RELATED APPLICATION(S)

This application claims the benefit of and priority to U.S. Provisional Application Ser. No. 63/592,452, entitled “DEVICE FOR ENDOSCOPIC IMAGING FOR ENDOSCOPIC PROCEDURES” and filed on Oct. 23, 2023, which is expressly incorporated by reference herein in its entirety.

### INTRODUCTION

A colonoscopy is an example of an endoscopy procedure including an examination of the large intestine or colon through the use of a colonoscope. A colonoscope is a flexible, tube-like inspection device having a camera at its end. Colonoscopies are performed for a variety of medical reasons including detection of inflamed tissue, ulcers, abnormal growths or polyps, and colorectal cancer. Colonoscopy is increasingly used as a screening tool to detect colorectal cancer.

During a colonoscopy, as an example of an endoscopy procedure, a colonoscope is inserted into a patient's rectum and then advanced to the beginning of the colon (an area known as the cecum) in order to examine the lining of the large intestine. The efficiency and accuracy of this procedure is largely dependent on the ease with which the colonoscope can be advanced. During the procedure, the colon may become over-distended or flopped in unnatural directions creating loops that hinder the advancement of the colonoscope and resulting in patient discomfort, longer examination times, and potentially inaccurate or incomplete screenings.

Currently, the difficulty in advancing the scope is addressed by the application of manual pressure by a technician to manually support the patient's colon. The application of manual pressure is time-consuming and varies depending on the particular technician's strength, technique, endurance, and training. In order to apply differential pressure or to change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure places a physical toll on the technician.

### SUMMARY

In an aspect of the disclosure, a method and apparatus for assisting a user in applying pressure to the abdomen of a patient and imaging of an endoscope to ease the passage of the endoscope during procedures used to examine the bowels including colonoscopy, sigmoidoscopy, and enteroscopy. Aspects presented herein provide a representation of the positioning and/or location of the endoscope on the abdomen of the patient to assist with the advancement, withdrawal, and/or visualization of the endoscope.

The aspects presented herein exert both broad, uniform lower abdominal pressure as well as provide information related to the positioning of the endoscope in relation to the colon including the sigmoid and transverse, and/or small bowel to assist with the advancement, withdrawal, and/or visualization as part of an endoscopy, colonoscopy, sigmoidoscopy, or enteroscopy procedure. Aspects presented herein provide improved visual tools to assist in preventing and reducing intestinal looping, eliminating the need for the application of manual pressure, improving patient safety, comfort, and satisfaction, and preventing musculoskeletal injury to endoscopy healthcare providers. Aspects may improve visualization and/or withdrawal portions of the procedure.

In some aspects, an apparatus to support an endoscopic procedure is provided. The apparatus includes an endoscopy device configured for placement at an abdomen of a patient during the endoscopic procedure, one or more sensors comprised in the endoscopy device and configured to obtain or provide information related to positioning of an endoscope in relation with a front portion of the endoscopy device or with a set of anatomical landmarks of the patient, and a visual indication component that changes visual output relative to a position of the endoscope.

In some aspects, the endoscopy device may be an endoscope compression device that is configured for the placement at/around the abdomen of the patient and is further configured to apply a pressure through compression on at least part of the abdomen of the patient.

In some aspects, a method is provided for support of an endoscopic procedure, with an endoscopy device configured for placement at an abdomen of a patient during the endoscopic procedure. The method includes obtaining or providing, in accordance with one or more sensors comprised in the endoscopy device, information related to positioning of an endoscope in relation with a front portion of the endoscopy device or with a set of anatomical landmarks of the patient, and changing, in accordance with a visual indication component of the endoscopy device, visual output relative to a position of the endoscope.

In some aspects, a non-transitory computer-readable medium storing computer executable code, for support of an endoscopic procedure, at an endoscopy device configured for placement at/around at abdomen of a patient during the endoscopic procedure is provided. The computer-readable medium includes code that when executed by processing circuitry causes the endoscopy device to: obtain or provide, in accordance with one or more sensors comprised in the endoscopy device, information related to positioning of an endoscope in relation with a front portion of the endoscopy device or with a set of anatomical landmarks of the patient; and change, in accordance with a visual indication component of the endoscopy device, visual output relative to a position of the endoscope.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

FIG. 1A is a schematic view of a colon with an endoscope (also known as a colonoscope for colonoscopy procedures) partially inserted therein.

FIG. 1B is a schematic view of a colon in which a sigmoid loop has developed due to an attempt to advance the endoscope against an unsupported colon wall.

FIG. 1C is a schematic view of a colon showing the application of manual pressure to the colon to facilitate insertion of an endoscope.

FIG. 2A is a front view of an endoscopy device or compression device, in accordance with aspects of the present invention.

FIG. 2B is a rear view of an endoscopy device or compression device, in accordance with aspects of the present invention.

FIG. 3A is a front view of an endoscopy device or compression apparatus, in accordance with aspects of the present invention.

FIG. 3B is a rear view of an endoscopy device or compression apparatus, in accordance with aspects of the present invention.

FIG. 4A is a front view of an endoscopy device or compression device, in accordance with aspects of the present invention.

FIG. 4B is a rear view of an endoscopy device or compression device, in accordance with aspects of the present invention.

FIG. 5 is a diagram of an imaging system, in accordance with aspects of the present invention.

FIG. 6 is a diagram of an imaging system, in accordance with aspects of the present invention.

FIG. 7 is a diagram of a visual representation of a scope during an endoscopic procedure.

FIG. 8 is a diagram of a visual representation of a scope during an endoscopic procedure.

FIG. 9A is a diagram of a visual representation of a scope during an endoscopic procedure, in accordance with aspects of the present invention.

FIG. 9B is a diagram of a visual representation of a scope during an endoscopic procedure, in accordance with aspects of the present invention.

FIG. 10 is a diagram of a visual representation of a scope during an endoscopic procedure, in accordance with aspects of the present invention.

FIG. 11 is a diagram of a computer system on which the disclosed system and method can be implemented, in accordance with various aspects of the present disclosure.

FIG. 12A and FIG. 12B illustrate example aspects of endoscopy devices that can be used in connection with various aspects of the present disclosure.

FIG. 13 is a flowchart of a method for support of an endoscopic procedure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

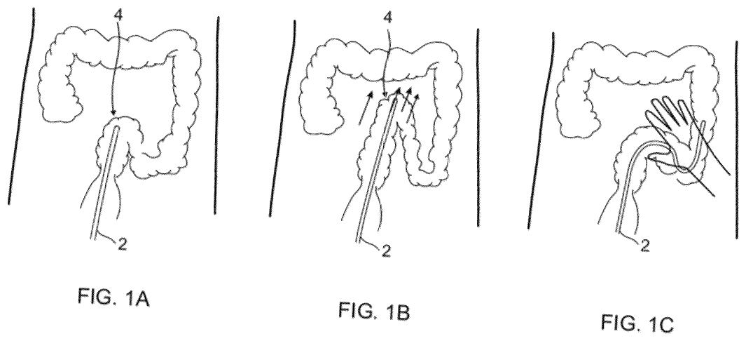

FIGS. 1A-1C, illustrate a sequence of steps of a colonoscopy, as one example of an endoscopy procedure. In FIG. 1A, a colonoscope 2 is inserted into the patient's rectum and advanced forward through the length of the colon. As the operator passes the colonoscope through the sigmoid region of the colon 4, the colonoscope may become impinged and cause distention and looping of the anatomy, as shown in FIG. 1B. The distention causes discomfort to the patient and increases the time required for the colonoscopy. In order to reduce the distended or looped area, a technician may apply manual pressure to the abdomen of the patient. Among other examples, the technician may be a nurse, assistant, or other staff member. For example, the pressure may be applied by a nurse or surgical assistant as shown in FIG. 1C.

The application of manual pressure is time-consuming and places a physical toll on the technician. The effectiveness of the manual pressure varies depending on the particular technician's strength, technique, endurance, and training. In order to apply differential pressure and change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure may lead to injury of the patient or of the technician.

Many patients undergo colonoscopy while placed in the left lateral decubitus position on the stretcher or operating table. Additional information about the use of such manual pressure can be found in Prechel J A, Hucke R. Safe and effective abdominal pressure during colonoscopy: forearm versus open hand technique. Gastroenterol Nurs 2009; 32:27-30; quiz 31-2, the entire contents of which are incorporated herein by reference. In applying manual pressure, the technician may reach over the patient from the opposite side of the table and to deploy pressure by placing their hands against the patient's sigmoid colon and then leaning backwards, using their bodyweight for leverage to exert force. While these methods are generally effective at generating pressure, they have also been identified as a causative factor for the high rate of work-related injuries among endoscopy nurses and staff. Physicians performing colonoscopy suffer work-related musculoskeletal injury at a particularly high-rate as well. The most frequent site of physician injury is the right upper extremity which experiences peak torque forces when while operators are attempting to advance the scope through (a looping) sigmoid colon. Additional details can be found in Spanarkel M, Hathorn J P. Looping During Colonoscopy: A Major, Implied Cause of Injury Among Endoscopy Healthcare Providers and a Proposed Solution, 2013, the entire contents of which are incorporated herein by reference.

Aspects presented herein assist the technician to more effectively provide targeted compression to a patient's abdomen during an endoscopy procedure. Aspects include visual components that are more intuitive for the technician to identify the location or position of the endoscopy within the patient's abdomen during an endoscopy procedure. By assisting the technician in more readily, or more accurately, identifying the position of the endoscope, the technician can more effectively apply targeted pressure during the procedure. The pressure may be applied manually or may be applied through adjustment of a compression device. In some aspects, a visual identification of the endoscope position may be provided at the patient's abdomen, such as at an LED array that is placed at the patient's abdomen. In some aspects, the visual identification may be provided at a display screen that shows the location of the endoscope relative to anatomical landmarks to enable the technician to more readily interpret the visual display and apply pressure at a corresponding location on the patient.

Aspects described herein may similarly be applied for other endoscopic procedures such as, but not limited to, sigmoidoscopy and retrograde enteroscopy procedures. Sigmoidoscopy is an examination of only the lower part of the colon, from the anus to the descending colon. An endoscope is inserted into the lower part of the colon. Enteroscopy is an examination of the small bowel. During retrograde enteroscopy, an endoscope is inserted in the anus and passed through the colon and the cecum and into the small bowel. Successfully navigating the loop-prone sigmoid region is necessary to complete both sigmoidoscopy and retrograde enteroscopy and thus aspects described herein can be used to help facilitate colonoscopy, sigmoidoscopy, retrograde enteroscopy, and other endoscopic procedures.

Each of the techniques can be used via an antegrade approach or a retrograde approach. In an antegrade approach, the scope is introduced through the patient's mouth and is advanced through the stomach in order to visualize the small intestine. In a retrograde approach, the scope is introduced through the rectum and through the colon and cecum in order to visualize the small intestine.

As described herein, one or more compression apparatuses can be applied at/around a patient enabling compression to be applied and/or adjusted at various points of the different procedures, e.g., as the scope is iteratively advanced deeper into the small bowel. The different levels and mechanisms of compression, presence/absence of compression, and/or targeted areas of compression may assist with the advancement of the scope, withdrawal of the scope, and/or visualization of the small bowel, colon, etc.

In order to apply effective compression and/or targeted pressure, it would be helpful for a user to know a location of the endoscope during the procedure, e.g., during insertion, visualization, and/or withdrawal. Aspects presented herein provide tools that assist the user in visualizing a location of the endoscope during an endoscopy procedure.

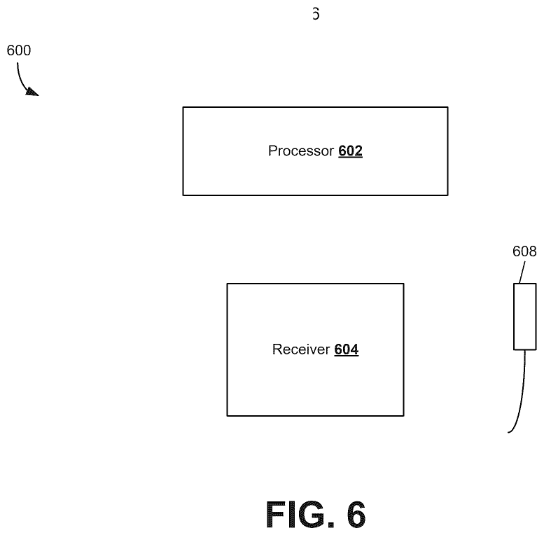

In some instances, as shown in diagram 600 of FIG. 6, a magnetic endoscopic imaging system may be utilized to assist in the visualization of the scope during the endoscopic procedure. The magnetic endoscopic imaging system may provide a representation of the position and/or configuration of the scope within the small bowel. The scope 2 may include electromagnetic coils along the length of the scope 608 that generate a pulsed low-intensity magnetic field that is detected by a receiver 604, where the receiver, scope, and coils interface with a processor 602. The low-intensity magnetic field is utilized to generate a representation of the position and configuration of the scope on a monitor. For example, the three-dimensional representation may be displayed on a monitor/display which may provide instant feedback as the scope is advanced during the procedure.

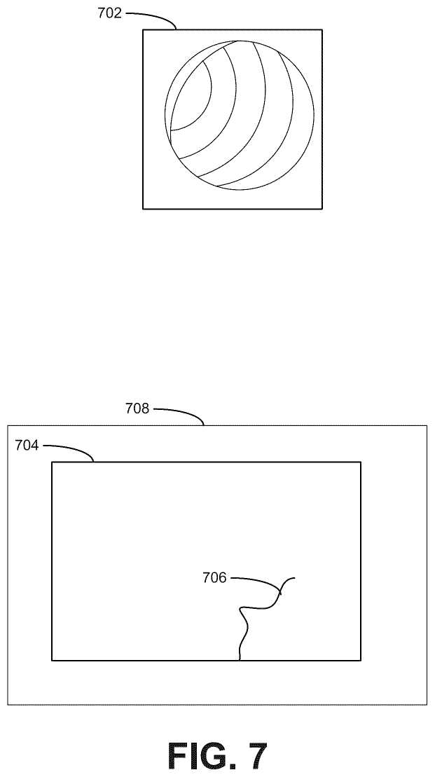

Although image orientation may be toggled or adjusted in conjunction with adjustments in patient body position (e.g. left lateral vs. supine vs. right lateral vs. prone) the magnetic endoscopic imaging system conveys limited information of the position of the scope. Aspects presented herein provide a system that improves user identification of scope location by rendering the image of scope shape and orientation onto the patient's body and/or convey the scope image relative to a set of common anatomical landmarks (also “marks” or “markers”). For example, the representation of the scope 706 location displayed on the monitor, as shown in FIG. 7, is shown in free space (e.g., 704) at a monitor 708 and does not include any reference points which may indicate the positioning of the scope 706 within the bowel 702 of a patient, which may limit the utility of the information being conveyed.

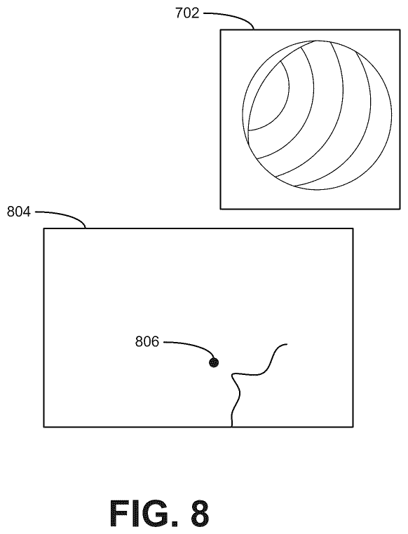

In some instances, as shown for example in FIG. 8, the magnetic endoscopic imaging system may provide an indication 806 on the display 804 where abdominal pressure intervention may be exerted to assist in the advancement of the scope. The indication 806 may correspond to the location of a device that is held by a user at the exterior of the abdomen of the patient. The indication 806 can be displayed on the display with respect to the three-dimensional representation of the scope. However, the utility of such indication to direct any type of abdominal pressure intervention may be limited or non-intuitive relative to the patient. It can be difficult for a practitioner to use the indication on the monitor to determine where the abdominal pressure intervention should be applied onto the patient's body.

Aspects presented herein provide an apparatus configured to convey a representation of the scope location with an image relative to a body outline and/or relative to common anatomical landmarks of the patient. Examples of such common anatomical landmarks include a marker that can be identified externally, such as a marker placed at the patient, or a body part such as the bones of the hip(s), pelvis, ribcage, spine, and/or the like. At least one advantage is that this would provide an enhanced representation of the scope with relation to the patient's body for scope location and looping location specific to the position of the actual patient. In some instances, the scope image may be shown relative to known anatomical features of the patient (e.g., skeletal structure, etc.) or relative to an outline of the patient's body.

Aspects presented herein may further provide an apparatus configured to provide the location of the scope and/or an indication of a location for abdominal pressure relative to the body outline and/or relative to common anatomical landmarks would provide an enhanced indication as to where to apply abdominal pressure. For example, the scope image and the indication of a location for abdominal pressure may be shown relative to known anatomical features of the patient (e.g., skeletal structure, etc.) or relative to an outline of the patient's body. In another example, the apparatus may be configured to show a representation of the shape and/or location of the scope onto the patient's body.

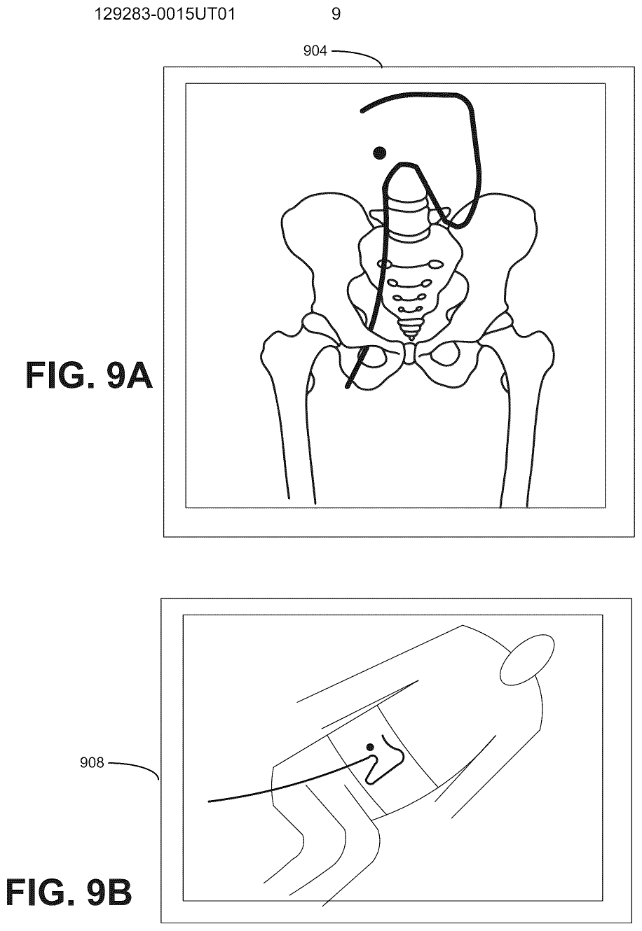

In some aspects, a colonoscopy device may be worn by or placed on a patient, where the colonoscopy device comprises one or more embedded coils and/or sensors that may also be identified by the receiver of the magnetic endoscopic imaging system. In some examples, the device may also be used as a compression device. The aspects to provide a visual indication of a location of an endoscope may also be incorporated into a non-compressive device that surrounds, or is placed over, at least a portion of the abdomen of the patient. The device may be referred to by any of various names, e.g., a compression device, an external device, an endoscopy device, an endoscopy visualization device, endoscopy visualization tool, an endoscopy landmark device, or an endoscopy marker device, among other examples. The one or more embedded coils and/or sensors may be configured to be aligned with specific areas on the device that may be aligned with specific anatomical locations of the patient. For example, the device may be placed with specific areas aligned with the patient's hips or a specific area aligned with the patient's navel. The device may be configured to remain in a fixed location at the patient during the procedure so that the information received from the sensors in the device enable a consistent determination of endoscope position relative to the device and therefore relative to the anatomical landmarks of the patient. This may allow the position and/or configuration of the scope to be conveyed (e.g., visually displayed) relative to the anatomical locations of the patient, which would enable an enhanced representation of the scope in relation with the patient's body, as shown for example in FIGS. 9A or 9B. For example, FIG. 9A illustrates an example display 904 that shows a position of the endoscope relative to an illustration of anatomical features. FIG. 9B illustrates an example of a display 908 that shows a position of the endoscope relative to an illustration of an outline of a patient.

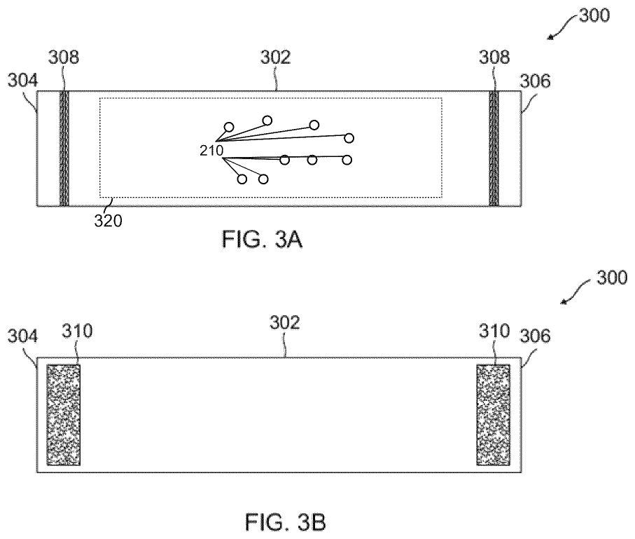

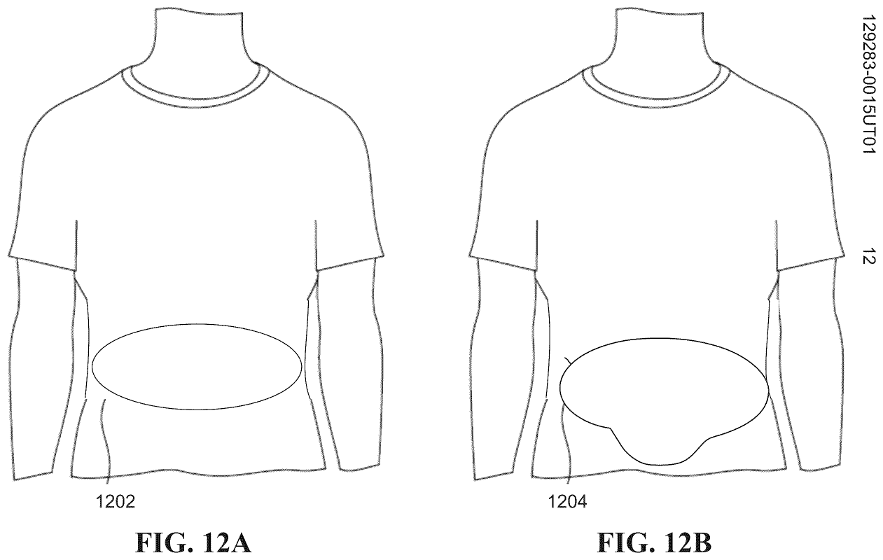

In some aspects, the endoscopy device (e.g., the compression apparatus 300 in FIG. 3) may comprise a wrap or a sleeve configured to surround at least a portion of an abdomen of the patient. In some aspects, the endoscopy device may include a layer that is placed over the patient's abdomen. The layer may include a rectangular layer, a rounded layer (e.g., as shown at 1202 in FIG. 12A), a shaped layer that is wider at the hips and narrower toward the bottom of the abdomen (e.g., as shown at 1204 in FIG. 12B), the layer may be shaped to cover a substantial portion of the abdomen. The device may be placed on or secured to the abdomen of the patient. In some aspects, at least a portion of the layer may adhere to the patient's abdomen. The layer may include the one or more sensors to detect a magnet at the endoscope and/or may include LEDs to provide a visual representation of the endoscope location within the patient. The device may comprise one or more sensors configured to provide information related to the position of the scope in relation with at least the front portion of the device. In some aspects, the device may be configured to apply pressure on at least part of the abdomen of the patient during an endoscopy procedure. In some aspects, the one or more sensors may detect one or more magnets of the scope as the scope traverses the bowel during the endoscopic procedure. In some aspects, the one or more sensors may provide the information related to the positioning of the scope in relation with anatomical landmarks of the patient. The one or more sensors may provide the information related to the positioning of the scope in relation with anatomical landmarks of the patient based at least on the one or more sensors being aligned with the anatomical landmarks of the patient. In some aspects, the positioning of the scope may be detected in relation to the one or more sensors.

In some aspects, a pressure indication may be provided in relation with the anatomical landmarks of the patient. For example, the pressure indication may indicate a location for external pressure on the abdomen of the patient to assist with the scope traversing the bowel of the patient.

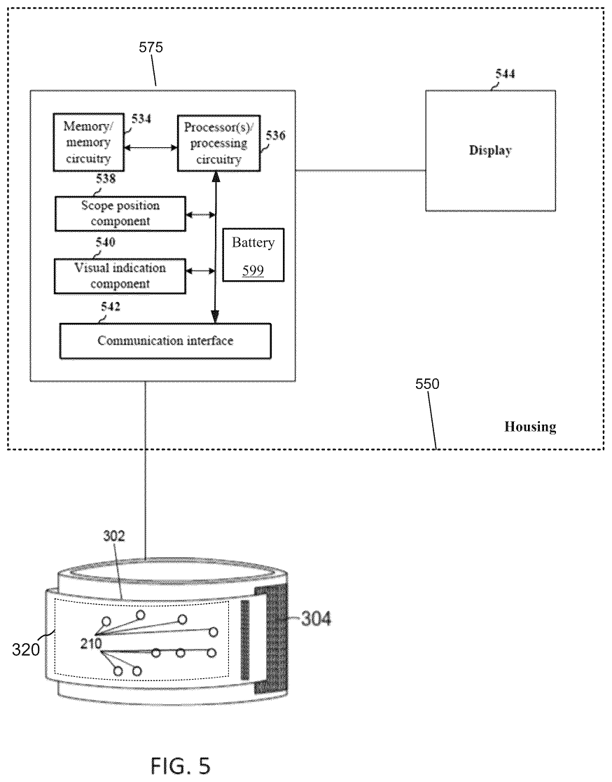

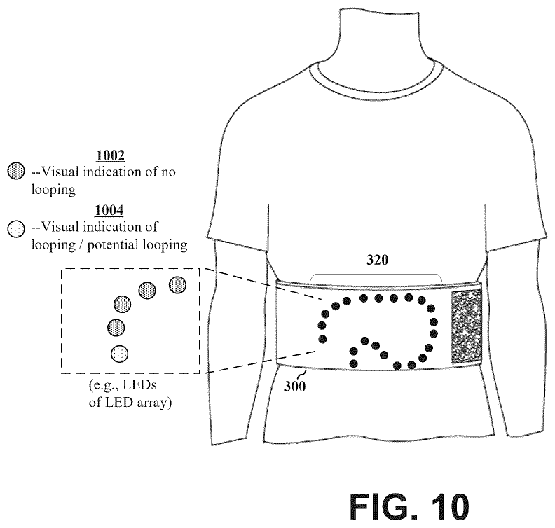

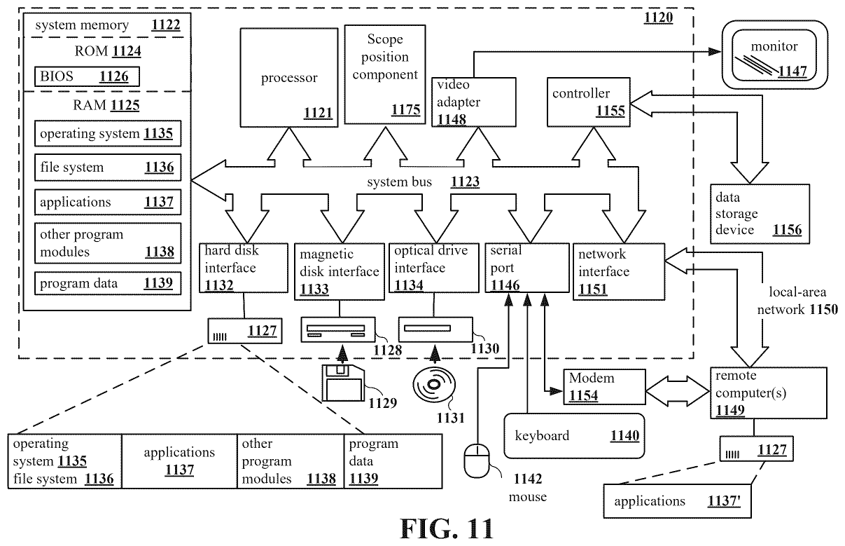

In some aspects, as shown for example in FIG. 10, the device may comprise a light emitting diode (LED) array. The aspects described in connection with FIG. 10 may be provided as an alternative to, or an addition to, the display aspects described in connection with FIGS. 9A and 9B. One or more LEDs of the LED array (e.g., the LED array 320 of the compression apparatus 300 in FIG. 3) may be activated (to emit light) to correspond with the position and a shape of the endoscope in response to the endoscope being proximate to the one or more LEDs of the LED array. For example, the LEDs may be activated by the magnetic field from the scope or may be activated by a signal emitted by the scope. In such instances, the sleeve may comprise a control unit comprising at least one processor coupled to at least one receiver and at least one transmitter. Example aspects of a control unit are illustrated in FIG. 5. The control unit may receive the signal emitted from the scope, as received by the one or more sensors, to detect, identify, or determine the positioning of the scope. FIG. 5 illustrates an example in which the control unit 575 may include memory or memory circuitry 534 and one or more processors or processing circuitry 536 configured to cause a visual indication of a position of an endoscope relative to a patient during an endoscopy procedure as described herein. In some aspects, the control unit 575 may be configured to cause the position of the endoscope to be displayed at the display 544 as described in connection with FIG. 9A and 9B. In some aspects, the control unit 575 may be configured to cause the position of the endoscope to be displayed in an LED array on the device at the abdomen of the patient, e.g., as described in connection with FIG. 10. In some aspects, the LED array may be part of a device, wrap, or sleeve that surrounds the patient, e.g., to hold the LED array in position at the abdomen of the patient. In some aspects, the control unit 575 may be provided at, comprised with, and/or removably attachable to the device (e.g., 302) that is configured to be positioned at/around the abdomen of the patient. The control unit 575 may provide a signal to the display 544 via a wired or wireless coupling via the communication interface 542. Although the display is shown as being within a housing 550, in some examples, in other examples, the display 544 may be outside of a housing that houses one or more other components of the control unit. In some aspects, the control unit 575 may be in a device that is separate from the device (e.g., 302) that is positioned at/around the patient. In such aspects, the control unit 575 may receive sensor information from and/or transmit control information (to control lights within the LED array) to the components provided at the device 302 via a wired or wireless coupling via the communication interface 542. In some aspects, the control unit 575 may be comprised in a housing 550 with the display 544. The control unit 575 may further include a battery 599 or power source. The control unit 575 may include a scope position component 538 that is configured to receive the sensor information via the communication interface 542 and translate the sensor information to a representation of the location of the endoscope within the patient, e.g., whether for display at a monitor and/or for display via an LED array 320 at the device 302. In some aspects, the scope position component 538 may receive a signal based on the position of the endoscope and/or based on a position of the device 302. The signal may be a reflected signal, e.g., based on a reflection of a signal transmitted by the scope position component 538. The signal may be a detection or reception of a pulsed signal provided by a component at the endoscope and/or the device 302. As an example, the endoscope may include a electro-magnetic coil component that can be detected (such as through pulses) by the scope position component 538. Similarly, the device 302 may include electro-magnetic component(s) that can be detected (such as through reception of pulses) by the scope position component 538. Thus, the endoscope and/or the device 302 may include a component that provides a signal to be received and interpreted by the scope position component 538, or the scope position component 538 may transmit a signal and may perform a measurement based on its own signal (e.g., such as a reflection) to determine information about the endoscope and/or device 302. By detecting the position of both the endoscope and the device 302, the scope position component may more accurately determine the location of the endoscope for display (e.g., either for display at the device 302 itself) and/or at a remote display using more detailed anatomical information. For example, the device 302 may be placed with a specific location relative to anatomical features of the patient, and the location of the device 302 gives a landmark or reference point for a determination of the scope position within the anatomy of the patient. In some aspects, the scope position component 538 may be included in the device 302, and may detect the location of the endoscope without a further detection of the location of the device 302. The control unit 575 may further include a visual indication component 540 that is configured to provide signals to the display 544 and/or LED array 320 to visually indicate the position of the endoscope relative to the anatomy of the patient. FIG. 11 illustrates an example of a processing system that includes additional aspects and components that may be included in or coupled to the control unit 575.

The positioning of the scope may be represented by the LED array, where the LED array is on or within the front portion of the sleeve. In some aspects, the LED array may be embedded within the front portion of the sleeve or compression device, such that one or more LEDs of the LED array may be activated in response to the scope traversing the bowel. In some aspects, the LED array may be embedded within one or more compression apparatuses or the sleeve. The device may extend over the abdomen of the patient and may be configured to apply a pressure upon at least a portion of the abdomen of the patient. For example, the device may include any of the aspects described in connection with U.S. Pat. No. 11,701,286 titled “Endoscopy Band with Sigmoid Support Apparatus” that issued on Jul. 18, 2023, the entire contents of which are incorporated herein by reference. The one or more compression apparatuses extending over the abdomen of the patient may allow for the LEDs of the LED array to be activated in response to the scope traversing the bowel.

In some aspects, the LED array may provide a visual indication 1002 to indicate an absence of looping and/or a visual indication 1004 to indicate that looping is likely to be occurring and/or to recommend that compression be applied to assist with movement of the endoscope. For example, the LED may illuminate with a first color to show the position of the endoscope when there is no looping, and may illuminate with a second color to indicate potential looping and/or to indicate a potential location for compression to assist with movement of the endoscope within the patient. The control unit, or another component of the device, may identify and/or predict looping based on the sensor information received from the one or more sensors, for example.

In some aspects, the control unit may be configured to transmit a positioning signal that indicates the positioning of the scope based on the signal from the scope. The receiver of the magnetic endoscopic imaging system may receive the positioning signal from the transmitter of the control unit to generate the three-dimensional image of the scope in relation with anatomical landmarks of the patient.

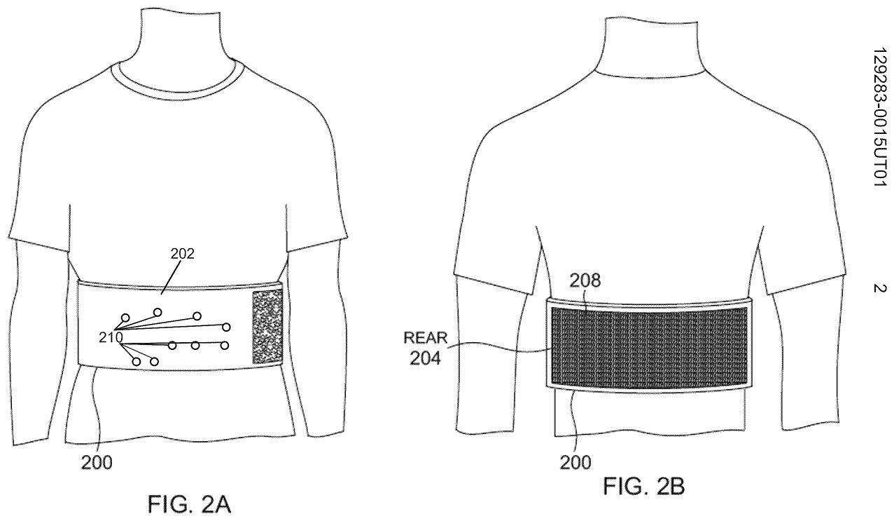

FIGS. 2A and 2B illustrate an example sleeve device. The sleeve 200 comprises a front portion 202, a rear portion 204, one or more anchor regions 208, and one or more sensors 210 on (e.g., as part of, attached to, etc.; generally “comprising”) at least the front portion 202 or the rear portion 204. Although referred to as a sleeve, the device may include a band that is configured to be fastened at/around the abdomen of the patient. The sleeve 200 is configured to surround at least a portion of the abdomen of the patient during endoscopic procedures (e.g., at least partially circumscribe the body of the patient while covering the abdomen, etc.). In some aspects, the sleeve 200 may act as a modular base that may be worn by patients, or placed under patients, in preparation of and/or during an endoscopy procedure that may require application of external and/or localized pressure to assist or allow the endoscope, or similar instrument, to advance the instrument during the procedure. The sleeve 200 may be configured as a modular base that may be worn by patients that may act as an anchoring base for one or more compression apparatuses that may deliver the external and/or localized pressure as desired. The sleeve may receive a variety of compression apparatuses, bands or attachments that may have different purposes. In some aspects, the sleeve may include a unitary section that encircles the abdomen of the patient. In some aspects, the sleeve may be a portion of a more comprehensive endoscopy garment.

The front portion 202 of the sleeve, when worn by the patient, may cover an abdominal region of the patient, while the rear portion 204 may cover a lower back region of the patient. The front portion 202 may be comprised of a first material that may have elastic properties. The first material of the front portion 202 may expand to accommodate various abdomen sizes of different patients to allow the sleeve to be utilized for patients of different body sizes. The rear portion 204 may be comprised of a second material that has less elasticity than that of the first material. In some aspects, the second material may be comprised of a semi-rigid or reinforced material. In some aspects, the second material may be comprised of one or more materials having different elasticities. For example, the second material may have a first elasticity along a rear central region of the rear portion 204, while the second material may have a second elasticity along the outer ends of the rear portion 204 that are proximate the front portion 202 of the sleeve. The first elasticity may be less elastic than the second elasticity, such that the second material having the first elasticity is maintained substantially aligned with a spine of the patient. The rear portion comprising the second material may maintain or align the rear portion 204 of the sleeve onto a back region of the patient. The rear portion 204 comprising the second material may be configured to provide a support structure to assist the one or more compression apparatuses to apply the pressure during the procedure. In some aspects, the rear portion 204 may counter pressure applied by the one or more compression apparatuses to maintain or align the rear portion 204 of the sleeve with the back region of the patient.

The one or more sensors 210 may be configured to be aligned with anatomical landmarks of the patient. The one or more sensors may be configured to obtain and/or provide information/indications related to the positioning of the scope in relation with at least the front portion 202 of the sleeve. In some aspects, the one or more sensors may obtain information about/detect one or more magnets of the scope in response to the scope traversing the bowel during the endoscopic procedure. In some aspects, the one or more sensors may detect the positioning of the scope in relation with anatomical landmarks of the patient. The one or more sensors may detect the positioning of the scope in relation with anatomical landmarks of the patient based at least on the one or more sensors being aligned with the anatomical landmarks of the patient, the one or more magnets of the scope, and/or one or more magnetic field pulses emitted from the scope.

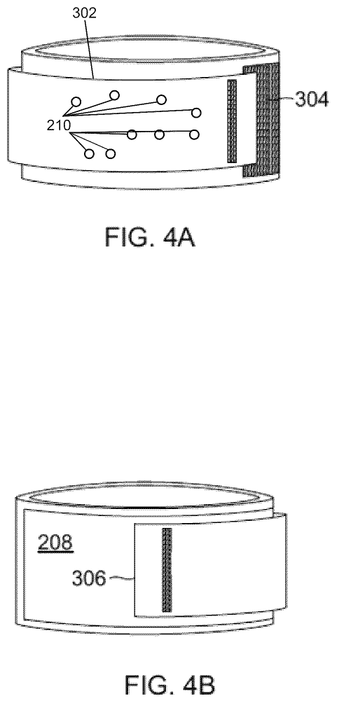

FIGS. 3A and 3B illustrate an example of an endoscopy device to be placed at the patient's abdomen, which may be configured as a compression apparatus 300. The compression apparatus 300 comprises a first end 304, a body of the device 302, and a second end 306. The first end 304 may be configured to be removably coupled to the front portion of the sleeve, as shown for example in FIG. 4A. The second end 306 may be configured to be removably coupled to the rear portion of the sleeve, such that the compression apparatus 300 may extend over the abdomen of the patient and apply the pressure upon at least a portion of the abdomen of the patient, as shown for example in FIGS. 4A and 4B.

The first end 304 of the compression apparatus 300 may comprise a fastener 310 such that the first end may couple to the rear portion 204 of the sleeve 200 (e.g., in FIG. 2B). For example, the fastener 310 may comprise a plurality of hooks while the rear portion of the sleeve comprises a plurality of loops, such that the plurality of hooks of the fastener 310 of the first end 304 may couple with the sleeve. In some aspects, the fastener 310 may comprise a plurality of loops while the front portion of the sleeve comprises a plurality of hooks, such that the first end may couple with the sleeve. The fastener 310 may comprise many different fastening devices and is not intended to be limited to a hook or loop configuration and may include snaps, adhesive, buttons, ties, or other fasteners. The fastener 310 is configured to correspond with the front portion 202 of the sleeve 200 such that the first end 304 may be removably coupled to the sleeve.

The second end 306 of the compression apparatus 300 may also comprise a fastener 310 configured in a similar manner as fastener 310 of the first end 304. The second end 306 of the compression apparatus 300 may be removably coupled to the rear portion of the sleeve. In some aspects, the second end of the compression apparatus may be removably coupled to at least the rear portion of the sleeve. The body of the device 302 of the compression apparatus 300 may extend over a portion of the abdomen of the patient and over part of the front portion of the sleeve, such that when the first end 304 is coupled to the front portion and the second end 306 is coupled to the rear portion while the body (e.g., 302) applies pressure on the portion of the abdomen of the patient. In some aspects, the compression apparatus 300 may comprise the one or more sensors 210, and may operate in a similar fashion as described above.

In some aspects, the one or more sensors 210 may comprise one or more LEDs of an LED array 320, such that the LED array 320 is comprised within the compression apparatus 300. In such aspects, one or more LEDs of the LED array 320 may be activated to correspond with the positioning and a shape of the scope in response to the scope being proximate to the one or more LEDs of the LED array 320. For example, the LEDs may be activated by the magnetic field from the scope or may be activated by a signal emitted by the scope. In such instances, the compression apparatus 300 may communicate with a control unit comprising at least one processor coupled to at least one receiver and at least one transmitter. The control unit may be configured receive a signal emitted from the scope as the scope traverses the bowel to detect the positioning of the scope. The positioning of the scope may be represented by the LED array 320, where the LED array 320 is arranged onto the front portion of the sleeve by the compression apparatus 300. In some aspects, the LED array 320 may be embedded within one or more compression apparatuses. The one or more compression apparatuses may be removably coupled to the sleeve and may comprise a first end and a body. The one or more compression apparatuses may extend over the abdomen of the patient to apply a pressure upon at least a portion of the abdomen of the patient. The one or more compression apparatuses extending over the abdomen of the patient may allow for the LEDs of the LED array 320 to be activated in response to the scope traversing the bowel.

In some aspects, the surface of the first anchor region may comprise a fastener that corresponds with a fastener on the first end of the compression apparatus, in order to removably couple the compression apparatus to the first anchor region. In some aspects, the first anchor region may comprise a plurality of anchor regions that collectively extend along at least a portion of the sleeve.

The first end 304 of the compression apparatus 300 may comprise the fastener 310 such that the first end may couple to the at least one attachment extension 206 of the sleeve 200. For example, the fastener 310 may comprise a plurality of hooks while the at least one attachment extension 206 comprises a plurality of loops, such that the plurality of hooks of the fastener 310 of the first end 304 may couple with the at least one attachment extension 206. In some aspects, the fastener 310 may comprise a plurality of loops while the at least one attachment extension 206 comprises a plurality of hooks, such that the first end may couple with the at least one attachment extension. The fastener 310 may comprise many different fastening devices and is not intended to be limited to a hook or loop configuration. The fastener 310 is configured to correspond with the attachment extension 206 of the sleeve 200 such that the first end 304 may be removably coupled to the attachment extension 206.

The second end 306 of the compression apparatus 300 may also comprise a fastener 310 configured in a similar manner as fastener 310 of the first end 304. The second end 306 of the compression apparatus 300 may be removably coupled to the one or more anchor regions 208. The body (e.g., 302) of the compression apparatus 300 may extend over a portion of the abdomen of the patient, such that when the first end 304 is coupled to the attachment extension 206 and the second end 306 is coupled to the one or more anchor regions 208, the body (e.g., 302) applies pressure on the portion of the abdomen of the patient.

In some aspects, the first end 304 or the second end 306 of the compression apparatus 300 may each comprise a handle 308. The handle 308 may be on the band opposite the fastener 310. The handle 308 at the first end 304 may be configured to assist with the placement or positioning of the first end 304 with the attachment extension 206. The handle 308 at the second end 306 may be configured to assist with the placement or positioning of the second end 306 with the anchor region 208.

Improving patient comfort and reducing complications, both during and following endoscopic procedures is very important. Aspects presented herein reduce patient discomfort and complications by helping to prevent and reduce sigmoid looping, which can be a primary cause of patient pain and discomfort.

To additionally enhance patient comfort, certain aspects of the invention may be designed to be single-use, and to remain fastened in place on the patient during the procedure and/or following the procedure. For example, maintaining the compression applied by the device during the withdrawal phase of the procedure and while imaging is performed may help improve the detection of adenoma. The device may be maintained on the patient to reduce the common post-procedure complications of bloating and abdominal pain caused by bloating. Otherwise known as gaseous distention, bloating occurs following endoscopy procedures because physicians often use compressed air or carbon dioxide to insufflate parts of the bowel that are difficult to see and examine. The gas opens up the area to allow for a more complete visualization, enhancing the efficacy of the procedure. However, the gas also remains in the patient until it is either absorbed or expelled. Expulsion is the primary gas removal mechanism as absorption is a very inefficient process. Gaseous distention is a primary post-procedure complication and a frequent complaint from patients. However, when the wrap described herein remains in place after the procedure, the lower abdominal compression generated by the device allows the bowel to more rapidly evacuate trapped by directing excess gas towards the rectum. As a result, the severity and duration of post-procedure bloating and associated abdominal pain may be reduced.

FIG. 11 is a block diagram illustrating a general-purpose computer system 1120 on which aspects of systems and methods for providing an improved display of a location of an endoscope within a patient during an endoscopy procedure, e.g., as described in connection with any of FIGS. 1-5, 9A, 9B, and 10 may be implemented in accordance with an example aspect.

As shown, the computer system 1120 (which may be a component of a personal computer or a server) includes a central processing unit 1121, a system memory 1122, and a system bus 1123 connecting the various system components, including the memory associated with the central processing unit 1121. As will be appreciated by those of ordinary skill in the art, the system bus 1123 may comprise a bus memory or bus memory controller, a peripheral bus, and a local bus that is able to interact with any other bus architecture. The system memory may include permanent memory (ROM) 1124 and random-access memory (RAM) 1125. The basic input/output system (BIOS) 1126 may store the basic procedures for transfer of information between elements of the computer system 1120, such as those at the time of loading the operating system with the use of the ROM 1124.

The computer system 1120 may also comprise a hard disk 1127 for reading and writing data, a magnetic disk drive 1128 for reading and writing on removable magnetic disks 1129, and an optical drive 1130 for reading and writing removable optical disks 1131, such as CD-ROM, DVD-ROM and other optical media. The hard disk 1127, the magnetic disk drive 1128, and the optical drive 1130 are connected to the system bus 1123 across the hard disk interface 1132, the magnetic disk interface 1133, and the optical drive interface 1134, respectively. The drives and the corresponding computer information media are power-independent modules for storage of computer instructions, data structures, program modules, and other data of the computer system 1120.

An example aspect comprises a system that uses a hard disk 1127 (which may store additional program applications 1137′), a removable magnetic disk 1129 and a removable optical disk 1131 connected to the system bus 1123 via the controller 1155. It will be understood by those of ordinary skill in the art that any type of media 1156 that is able to store data in a form readable by a computer (solid state drives, flash memory cards, digital disks, random-access memory (RAM) and so on) may also be utilized.

The computer system 1120 has a file system 1136, in which the operating system 1135 may be stored, as well as program applications 1137, other program modules 1138, and program data 1139. A user of the computer system 1120 may enter commands and information using keyboard 1140, mouse 1142, or any other input device known to those of ordinary skill in the art, such as, but not limited to, a microphone, joystick, game controller, scanner, etc. Such input devices typically plug into the computer system 1120 through a serial port 1146, which in turn is connected to the system bus, but those of ordinary skill in the art will appreciate that input devices may be also be connected in other ways, such as, without limitation, via a parallel port, a game port, or a universal serial bus (USB). A monitor 1147 or other type of display device may also be connected to the system bus 1123 across an interface, such as a video adapter 1148. In addition to the monitor 1147, the personal computer may be equipped with other peripheral output devices (not shown), such as loudspeakers, a printer, etc.

Computer system 1120 may operate in a network environment, using a network connection to one or more remote computers 1149. The remote computer (or computers) 1149 may be local computer workstations or servers comprising most or all of the aforementioned elements in describing the nature of a computer system 1120. Other devices may also be present in the computer network, such as, but not limited to, routers, network stations, peer devices or other network nodes.

Network connections can form a local-area computer network (LAN) 1150 and a wide-area computer network (WAN). Such networks are used in corporate computer networks and internal company networks, and they generally have access to the Internet. In LAN or WAN networks, the computer system 1120 is connected to the local-area network 1150 across a network adapter or network interface 1151. When networks are used, the computer system 1120 may employ a modem 1154 or other modules well known to those of ordinary skill in the art that enable communications with a wide-area computer network such as the Internet. The modem 1154, which may be an internal or external device, may be connected to the system bus 1123 by a serial port 1146. It will be appreciated by those of ordinary skill in the art that said network connections are non-limiting examples of numerous well-understood ways of establishing a connection by one computer to another using communication modules.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

In various aspects, the systems and methods described in the present disclosure can be addressed in terms of modules. The term “module” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module's functionality, which (while being executed) transform the microprocessor system into a special-purpose device. A module, element, or component may also be implemented as a combination of the two, with particular functions facilitated by hardware alone, and other functions facilitated by a combination of hardware and software. In particular implementations, at least a portion, and in some cases, all, of a module, element, or component may be executed on one or more processors of a general purpose computer. Accordingly, each module may be realized in a variety of suitable configurations, and should not be limited to any particular implementation or example herein. An element, or any portion of an element, or any combination of elements may be implemented as a “processing system” that includes one or more processors. When multiple processors are implemented, the multiple processors may perform the functions individually or in combination. One or more processors in a processing system may execute stored instructions, which may be referred to as software, firmware, middleware, microcode, hardware description language, or otherwise, e.g., instructions, instruction sets, code, code segments, program code, programs, subprograms, software components, applications, software applications, software packages, routines, subroutines, objects, executables, threads of execution, procedures, functions, or any combination thereof.

In one configuration, the scope position component 1175 and/or the computer system 1120, and in particular, the file system 1136 and/or the processor (e.g., 1121), is configured to perform the aspects of any of FIGS. 1-5, 9A, 9B, and 10. In some aspects, the scope position component 1175 may correspond to one or more of the scope position component 538 and/or the visual indication component 540 of the control unit 575 illustrated in FIG. 5.

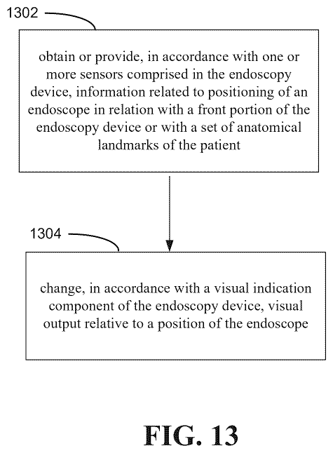

FIG. 13 illustrates an example flowchart showing a method for support of an endoscopic procedure, with an endoscopy device configured for placement at an abdomen of a patient during the endoscopic procedure. As shown at 1302, the method includes obtaining or providing, in accordance with one or more sensors comprised in the endoscopy device, information related to positioning of an endoscope in relation with a front portion of the endoscopy device or with a set of anatomical landmarks of the patient. The endoscopy device may include any of the aspects described in connection with FIGS. 1-12B. In some aspects, the endoscopy device may be a compression device.

At 1304, the method includes changing, in accordance with a visual indication component of the endoscopy device, visual output relative to a position of the endoscope. In some aspects, changing the visual output includes: activating one or more LEDs of an LED array provided on the endoscopy device to correspond with the relative position and a shape of the endoscope. In some aspects, changing the visual output includes outputting to a display a visual indication of the position of the endoscope relative to the set of anatomical landmarks displayed at the display, and wherein the display is separate from the endoscopy device, and wherein the endoscopy device includes a reference component that provides a reference for the anatomical landmarks relative to the positioning of the endoscope.

In some aspects, the method may further include detecting, via the one or more sensors, one or more magnets of the endoscope during the endoscopic procedure; and identifying, based on information from the one or more sensors, the relative position of the endoscope relative to the front portion of the endoscopy device or relative to the set of anatomical landmarks of the patient.

In some aspects, the one or more sensors are configured to detect the one or more magnets of the endoscope as the endoscope traverses a colon of the patient. In some aspects, the one or more sensors are configured to provide the information related to the positioning of the endoscope in relation with the set of anatomical landmarks of the patient. In some aspects, the method includes aligning the one or more sensors are with the set of anatomical landmarks of the patient, and wherein the positioning of the endoscope is detected in relation to the one or more sensors.

In some aspects, the method includes providing a visual pressure indication in relation with the set of anatomical landmarks of the patient, wherein the pressure indication indicates a location for external pressure on the abdomen of the patient.

In some aspects, the visual indication component a LED array provided on the endoscopy device, wherein one or more LEDs of the LED array are configured to activate to correspond with the relative position and a shape of the endoscope. In some aspects, the method includes activating in a visual indication, associated with the relative position of the endoscope, to correspond with at least one of: a presence or a potential presence of looping, or a location to apply a pressure through compression on at least part of the abdomen of the patient.

In some aspects, the endoscopy device is a compression device, and the method further includes applying the pressure through the compression through application of the device on at least part of the abdomen of the patient that corresponds to the location and the visual indication. In some aspects, the method includes activating the one or more LEDs of the LED array in a different visual indication, associated with the positioning of the endoscope, to correspond with an absence of looping.

In some aspects, the method includes outputting to a display a visual indication of the position of the endoscope relative to an outline of the patient displayed at the display, wherein the display is separate from the endoscopy device.

Example aspects of the present invention have now been described in accordance with the above advantages. It will be appreciated that these examples are merely illustrative of aspects of the present invention. Many variations and modifications will be apparent to those skilled in the art.

It is understood that the specific order or hierarchy of steps in the processes disclosed is an illustration of example approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects.” Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

The following aspects are illustrative only and may be combined with other aspects or teachings described herein, without limitation.

Aspect 1 is an apparatus to support an endoscopic procedure, comprising: an endoscopy device configured for placement at an abdomen of a patient during the endoscopic procedure; one or more sensors comprised in the endoscopy device and configured to obtain or provide information related to positioning of an endoscope in relation with a front portion of the endoscopy device or with a set of anatomical landmarks of the patient; and a visual indication component that changes visual output relative to a position of the endoscope.

Aspect 2 is the apparatus of aspect 1, wherein the endoscopy device is an endoscope compression device that is configured for the placement at/around the abdomen of the patient and is further configured to apply a pressure through compression on at least part of the abdomen of the patient.

Aspect 3 is the apparatus of any of aspects 1 and 2, wherein the one or more sensors are configured to detect one or more magnets of the endoscope during the endoscopic procedure, wherein the apparatus further includes one or more processors configured to: identify, based on information from the one or more sensors, the relative position of the endoscope relative to the front portion of the endoscopy device or relative to the set of anatomical landmarks of the patient.

Aspect 4 is the apparatus of aspect 3, wherein the one or more sensors are configured to detect the one or more magnets of the endoscope as the endoscope traverses a colon of the patient.

Aspect 5 is the apparatus of any of aspects 1 to 4, wherein the one or more sensors are configured to provide the information related to the positioning of the endoscope in relation with the set of anatomical landmarks of the patient.

Aspect 6 is the apparatus of aspect 5, wherein the one or more sensors are aligned with the set of anatomical landmarks of the patient, and wherein the positioning of the endoscope is detected in relation to the one or more sensors.

Aspect 7 is the apparatus of aspect 5, wherein the visual indication component is configured to provide a visual pressure indication in relation with the set of anatomical landmarks of the patient, wherein the pressure indication indicates a location for external pressure on the abdomen of the patient.

Aspect 8 is the apparatus of aspect 5, wherein the visual indication component comprises: a light emitting diode (LED) array provided on the endoscopy device, wherein one or more LEDs of the LED array are configured to activate to correspond with the relative position and a shape of the endoscope.

Aspect 9 is the apparatus of aspect 8, wherein the one or more LEDs of the LED array are configured to activate in a visual indication, associated with the relative position of the endoscope, to correspond with at least one of: a presence or a potential presence of looping, or a location to apply a pressure through compression on at least part of the abdomen of the patient.

Aspect 10 is the apparatus of aspect 9, wherein the endoscopy device is an endoscope compression device that is further configured to apply the pressure through the compression on at least part of the abdomen of the patient that corresponds to the location and the visual indication.

Aspect 11 is the apparatus of aspect 9, wherein the one or more LEDs of the LED array are configured to activate in a different visual indication, associated with the positioning of the endoscope, to correspond with an absence of looping.

Aspect 12 is the apparatus of any of aspects 1 to 11, wherein the visual indication component is configured to output to a display a visual indication of the position of the endoscope relative to the set of anatomical landmarks displayed at the display, and wherein the display is separate from the endoscopy device, and wherein the endoscopy device includes a reference component that provides a reference for the anatomical landmarks relative to the positioning of the endoscope.

Aspect 13 is the apparatus of any of aspects 1 to 12, wherein the visual indication component is configured to output to a display a visual indication of the position of the endoscope relative to an outline of the patient displayed at the display, wherein the display is separate from the endoscopy device.

Aspect 14 is the apparatus of any of aspects 1 to 13, wherein the endoscopy device further comprises a control unit comprising at least one processor coupled to at least one receiver and at least one transmitter, wherein the control unit is configured to receive a signal from the endoscope via the one or more sensors to detect the positioning of the endoscope.

Aspect 15 is the apparatus of aspect 14, wherein the control unit is configured to provide a positioning signal that indicates the positioning of the endoscope based on the signal from the endoscope.

Aspect 16 is the apparatus of aspect 15, wherein the control unit is configured to provide the positioning signal for at least one of a display or a light emitting diode (LED) array.

Aspect 17 is a method for support of an endoscopic procedure, with an endoscopy device configured for placement at an abdomen of a patient during the endoscopic procedure, comprising: obtaining or providing, in accordance with one or more sensors comprised in the endoscopy device, information related to positioning of an endoscope in relation with a front portion of the endoscopy device or with a set of anatomical landmarks of the patient; and changing, in accordance with a visual indication component of the endoscopy device, visual output relative to a position of the endoscope.

Aspect 18 is the method of aspect 17, wherein changing the visual output includes: activating one or more light emitting diodes (LEDs) of an LED array provided on the endoscopy device to correspond with the relative position and a shape of the endoscope.

Aspect 19 is the method of any of aspects 17 and 18, wherein changing the visual output includes: outputting to a display a visual indication of the position of the endoscope relative to the set of anatomical landmarks displayed at the display, and wherein the display is separate from the endoscopy device, and wherein the endoscopy device includes a reference component that provides a reference for the anatomical landmarks relative to the positioning of the endoscope.

Aspect 20 is a method to support an endoscopic procedure, comprising performing operations in accordance with the apparatus of any of aspects 1 to 16.

Aspect 21 is a computer-readable medium (e.g., a non-transitory computer-readable medium) storing computer executable code at an endoscopy device, the code when executed by at least one processor causes the at least one processor to perform operations in accordance with the apparatus of any of aspects 1 to 16.

Aspect 22 is an apparatus to support an endoscopic procedure, comprising: at least one memory; and at least one processor coupled to the at least one memory, the at least one processor, individually or in any combination, is configured to perform the method of any of aspects 17 to 19.

Aspect 23 is an apparatus to support an endoscopic procedure, comprising means for performing each step in the method of any of aspects 17 to 19.

Aspect 24 is a computer-readable medium (e.g., a non-transitory computer-readable medium) storing computer executable code at an endoscopy device, the code when executed by at least one processor causes the at least one processor to perform the method of any of aspects 17 to 19.

1. An apparatus to support an endoscopic procedure, comprising:
an endoscopy device configured for placement at an abdomen of a patient during the endoscopic procedure;
one or more sensors comprised in the endoscopy device and configured to obtain or provide information related to positioning of an endoscope in relation with a front portion of the endoscopy device or with a set of anatomical landmarks of the patient; and
a visual indication component that changes visual output relative to a position of the endoscope.

2. The apparatus of claim 1, wherein the endoscopy device is an endoscope compression device that is configured for the placement at the abdomen of the patient and is further configured to apply a pressure through compression on at least part of the abdomen of the patient.

3. The apparatus of claim 1, wherein the one or more sensors are configured to detect one or more magnets of the endoscope during the endoscopic procedure, wherein the apparatus further includes one or more processors configured to:
identify, based on information from the one or more sensors, the relative position of the endoscope relative to the front portion of the endoscopy device or relative to the set of anatomical landmarks of the patient.

4. The apparatus of claim 3, wherein the one or more sensors are configured to detect the one or more magnets of the endoscope as the endoscope traverses a colon of the patient.

5. The apparatus of claim 1, wherein the one or more sensors are configured to provide the information related to the positioning of the endoscope in relation with the set of anatomical landmarks of the patient.

6. The apparatus of claim 5, wherein the one or more sensors are aligned with the set of anatomical landmarks of the patient, and wherein the positioning of the endoscope is detected in relation to the one or more sensors.

7. The apparatus of claim 5, wherein the visual indication component is configured to provide a visual pressure indication in relation with the set of anatomical landmarks of the patient, wherein the pressure indication indicates a location for external pressure on the abdomen of the patient.

8. The apparatus of claim 5, wherein the visual indication component comprises:
a light emitting diode (LED) array provided on the endoscopy device, wherein one or more LEDs of the LED array are configured to activate to correspond with the relative position and a shape of the endoscope.

9. The apparatus of claim 8, wherein the one or more LEDs of the LED array are configured to activate in a visual indication, associated with the relative position of the endoscope, to correspond with at least one of:
a presence or a potential presence of looping, or
a location to apply a pressure through compression on at least part of the abdomen of the patient.

10. The apparatus of claim 9, wherein the endoscopy device is an endoscope compression device that is further configured to apply the pressure through the compression on at least part of the abdomen of the patient that corresponds to the location and the visual indication.

11. The apparatus of claim 9, wherein the one or more LEDs of the LED array are configured to activate in a different visual indication, associated with the positioning of the endoscope, to correspond with an absence of looping.

12. The apparatus of claim 1, wherein the visual indication component is configured to output to a display a visual indication of the position of the endoscope relative to the set of anatomical landmarks displayed at the display, and wherein the display is separate from the endoscopy device, and wherein the endoscopy device includes a reference component that provides a reference for the anatomical landmarks relative to the positioning of the endoscope.

13. The apparatus of claim 1, wherein the visual indication component is configured to output to a display a visual indication of the position of the endoscope relative to an outline of the patient displayed at the display, wherein the display is separate from the endoscopy device.

14. The apparatus of claim 1, wherein the endoscopy device further comprises a control unit comprising at least one processor coupled to at least one receiver and at least one transmitter, wherein the control unit is configured to receive a signal from the endoscope via the one or more sensors to detect the positioning of the endoscope.

15. The apparatus of claim 14, wherein the control unit is configured to provide a positioning signal that indicates the positioning of the endoscope based on the signal from the endoscope.

16. The apparatus of claim 15, wherein the control unit is configured to provide the positioning signal for at least one of a display or a light emitting diode (LED) array.

17. A method for support of an endoscopic procedure, with an endoscopy device configured for placement at an abdomen of a patient during the endoscopic procedure, comprising:
obtaining or providing, in accordance with one or more sensors comprised in the endoscopy device, information related to positioning of an endoscope in relation with a front portion of the endoscopy device or with a set of anatomical landmarks of the patient; and
changing, in accordance with a visual indication component of the endoscopy device, visual output relative to a position of the endoscope.

18. The method of claim 17, wherein changing the visual output includes:
activating one or more light emitting diodes (LEDs) of an LED array provided on the endoscopy device to correspond with the relative position and a shape of the endoscope.

19. The method of claim 17, wherein changing the visual output includes:
outputting to a display a visual indication of the position of the endoscope relative to the set of anatomical landmarks displayed at the display, and wherein the display is separate from the endoscopy device, and wherein the endoscopy device includes a reference component that provides a reference for the anatomical landmarks relative to the positioning of the endoscope.

20. A non-transitory computer-readable medium storing computer executable code, for support of an endoscopic procedure, at an endoscopy device configured for placement at an abdomen of a patient during the endoscopic procedure, the code when executed by processing circuitry causes the endoscopy device to:
obtain or provide, in accordance with one or more sensors comprised in the endoscopy device, information related to positioning of an endoscope in relation with a front portion of the endoscopy device or with a set of anatomical landmarks of the patient; and
change, in accordance with a visual indication component of the endoscopy device, visual output relative to a position of the endoscope.
